(19) United States
(12) Patent Application Publication
(10) Pub. No.: US 2009/0228082 A1
(43) Pub. Date: Sep. 10, 2009

(54) Patient heat transfer device

(75) Inventors: Francis L. Ross, III, McLean, VA (US); Gary Searle, Norfolk, MA (US)

Correspondence Address: LOUIS WOO;LAW OFFICE OF LOUIS WOO, 717 NORTH FAYETTE STREET, ALEXANDRIA, VA, 22314, US

(73) Assignee: Smiths Medical ASD, Inc., Rockland, MD (US)

(21) Appl. No.: 12/379,756

(22) Filed: Feb. 27, 2009

Related U.S. Application Data
(60) Provisional application No. 61/064,463, filed on Mar. 7, 2008.

(51) Int. Cl. A61F 7/02

(52) U.S. Cl. 607/107; 607/104

(57) ABSTRACT

To rapidly induce hypothermia to a patient, in the event the patient has a stroke, hyperthermia or some other temperature related heath problems which requires prompt action to regulate the temperature of the patient, a flat flexible structure conformable to the body of the patient is placed into contact with the patient. The structure has at least two heat transfer portions. One of heat transfer portions is positioned in contact with the body of the patient. The structure is hermetically sealed and a fluidized medium responsive to temperature change is provided in the structure between the heat transfer portions. The fluid is changeable between a liquid state and a gaseous state, when it is exposed to heat and cold. The heat absorbed by the heat transfer portion in contact with the patient is carried by the fluidized medium, as latent heat in the gas that results when the liquid is vaporized to its gaseous state, to the heat transfer portion layer not in contact with the patient, so that the latent heat in the gaseous vapor is dissipated. Upon dissipation, the gaseous vapor is condensed and the fluidized medium returns to its liquid state. The structure may be formed from a flat flexible heat pipe and may also be configured as a rib cage shaped jacket to embrace the torso of the patient. A cooling circuit or mechanism may be added to the structure to facilitate the removal of heat therefrom. The structure may also be used to raise the core temperature of a patient.

### FIELD OF THE INVENTION

The present invention relates to devices for transferring thermal energy to and from a patient, and more particularly a device that may be in the form of a cover, a sheet or a pad placable over or under a patient that is adaptable to at least induce hypothermia to the patient.

### BACKGROUND OF THE INVENTION

It has been found that for stroke victims as well as patients with other medical conditions such as hyperthermia that lowering the body temperature of the patient such as by induced hypothermia lowers the risk of permanent damage to the patient and increases his survival rate. There are two main methods of cooling: external cooling and invasive cooling. Invasive cooling uses seemingly extreme but effective measures such as passing the blood or saline to be input to a patient through a heat exchanger. Less invasive methods involve injecting chilled saline solution. External cooling uses a device external to the patient to cool the body temperature of the patient and is therefore much easier to apply and inherently more attractive in the medical setting, as it does not require a high degree of technical expertise or careful monitoring. If the device is portable then it could even be applied out in the field. Virtually all existing external cooling devices require a compressor unit that circulates chilled water.

One external cooling device is disclosed in U.S. Pat. No. 6,197,045 where a thermal exchange fluid is circulated through a fluid layer of a medical pad to conductively remove heat from the patient in contact with the pad. The fluid is drawn into the pad by the negative pressure created by an external pump connected downstream from the outlet of the pad. A fluid reservoir connected upstream from the inlet of the pad completes the fluid path to enable the fluid to circulate into and out of the pad. As the fluid is drawn through the fluid circulating layer of the pad, heat is exchanged between the patient and the fluid. Such circulation of a thermal exchange fluid through a pad via an inlet and an outlet to the pad is considered “active external cooling”.

Invasive cooling delivers faster cooling than the active external cooling device disclosed in the '045 patent. However, such cooling requires that the patient be catheterized.

### SUMMARY OF THE PRESENT INVENTION

The instant invention relates to a self contained external temperature regulating device that enables controlled and rapid induced hypothermia for stroke victims and other patients who require that their body temperature be non-invasively lowered, and controlled non-invasive increase in the body temperature for those patients whose body temperature has become too low and would need to be normalized. The normal body temperature of the patient may be controllably restored after the patient has been subjected to the induced hypothermia.

It has been determined through thermal analysis that the thermal resistance of the flesh and skin dictates the cooling rate of a person, and that the selection of materials is not important as long as it is flexible and conformal with the body of the person so as to avoid air pockets. One of the inventors has also determined that an efficient way to transfer thermal energy, i.e., heat, between a patient and an external device is for that device to be a flexible material that can cover or be draped over at least one portion of the body of the patient, or placed underneath at least one portion of the patient. A requirement is that the portion of the device that transfers heat to or from the patient be in contiguous or intimate contact with the desired portion of the body of the patient, so that the transfer of heat between the patient and the device be enhanced. It has furthermore been determined that a basic polyolefin laminate with a pressure sensitive adhesive (PSA) may be used for contactedly securing the flexible material to the patient.

To that end, the instant invention may employ a heat pipe, more specifically a flat flexible heat pipe that is conformal to the human body. A heat pipe is a two phase heat transfer device that has a high effective thermal conductivity. The heat pipe utilized for the instant invention may be a flat flexible structure that is conformable to the shape of the body of the patient. The structure is hermetically sealed with at least two heat transfer portions. One of the heat transfer portions may be in the form of a layer placed in intimate or contiguous contact with at least a portion of the body of the patient, and functions to transfer the thermal energy between the patient and the device. The other heat transfer portion, which may also be a layer, does not come into contact with the patient, and functions as a heat dissipater or condenser through which heat is removed from the structure. A fluidized medium that changes physical phases dependent on the temperature it encounters is provided inside the structure for moving the thermal energy between the two heat transfer portions. Thus, to induce hypothermia in the patient, the heat from the patient, transferred to the heat transfer portion he is in contact with, is carried by the fluidized medium as latent heat in a gaseous state to the heat transfer portion that acts as a heat dissipater or condenser for dissipating the latent heat and condenses the gaseous fluidized medium back into a liquid. A structure such as a flat flexible heat pipe provides an excellent device to effect an isothermal process or condition as thermal energy is equalized across its entire surface.

To enhance the removal of heat from the structure, and hence from the patient, a cooling system that may in the form of a chiller circuit is connected to the heat dissipation or condenser portion of the structure so that whatever heat collected at that portion may be conductively removed by the chiller fluid. Cooled water may be used as the circulation medium for cooling in the chiller circuit. In place of a chiller circuit, a cooling medium such as air may be provided to the heat dissipation portion of the structure to enhance the dissipation of heat therefrom. In which case, as the temperature of the heat dissipation portion is dependent on the ambient temperature, the temperature at the heat dissipation portion needs to be at a lower temperature than the temperature at the heat transfer portion in contact with the patient. To facilitate heat input to the patient in the case of normalizing the body temperature of the patient from a low body temperature, a heating system in the form of a heating circuit that uses a heated circulation fluid medium such as water may be connected to the condenser portion of the structure to transfer heat to the structure.

To ensure that the heat transfer portion that comes into contact with the patient be in continuous and intimate contact with the patient, an adhesive layer is provided on the surface of the structure that forms the heat transfer portion and comes into contact with the patient, so that once pressed onto the patient, the surface of the heat transfer portion of the structure will remain in contiguous and intimate contact with the body of the patient, without any air pockets between the contact area of the structure and the patient.

The fluidized medium provided in the structure may be water, ethanol, methanol or some other similar medium, under vacuum in the structure, that changes from a liquid to a gaseous phase or state when exposed to heat. In addition, a wick layer that forms grooves or a layer of porous sintered metal may be provided in the structure to enable capillary action in the structure for the fluidized medium, when vaporized from its liquid state to its gaseous state and carrying the latent heat, to traverse between the two heat transfer portions of the structure.

The present invention is therefore a device that is placable over or under at least a portion of a patient for therapeutically cooling the patient. The device includes a structure conformable to the shape of the body of the patient, with the structure being hermetically sealed and having at least one heat transfer portion in contact with the patient, at least one heat dissipation portion and a fluidized medium changeable between a liquid state and a gaseous state provided in the structure for moving the heat transferred from the patient to the heat transfer portion to the heat dissipation portion for removal. The device further includes an adhesive layer provided on the patient contact surface of the structure to ensure that the structure is in contiguous contact with the patient.

The instant invention also is a therapeutic device placable over or under a patient for regulating the body temperature of the patient that comprises a structure substantially conformable to the shape of the body of a patient, with the structure being hermetically sealed and having a first thermal energy transfer portion in contact with the patient, a second thermal energy transfer portion and a fluidized medium within the structure that is changeable between a liquid state and a gaseous state for carrying heat absorbed from one of the thermal energy transfer portions to the other of the thermal energy transfer portions. The therapeutic device further includes an adhesive layer provided on the patient contact surface of the structure to ensure that at least the first energy transfer portion of the structure is in contiguous contact with the patient.

The instant invention further relates to a system for therapeutically regulating the body temperature of the patient which includes a structure substantially conformable to the shape of the body of the patient that is hermetically sealed and having at least one heat transfer portion in contact with the patient, at least another heat transfer portion and a fluidized medium changeable between a liquid state and a gaseous state for moving heat absorbed at one of the heat transfer portions to the other of the heat transfer portions for removal, and an adhesive layer provided on the patient contact surface of the structure to ensure that the heat transfer portion in contact with patient remains in contact with the patient.

The instant invention moreover relates to a method of therapeutically regulating the body temperature of the patient that includes the step of placing a structure substantially conformable to the shape of the body of the patient into contact with the patient, the structure being hermetically sealed and having a first thermal energy transfer portion that comes into contact with the patient, a second thermal energy transfer portion and a fluidized medium changeable between a liquid state and a gaseous state within the structure for carrying the heat absorbed from one of the thermal energy transfer portion to the other of the thermal energy transfer portion, and the step of adhering the first thermal energy transfer portion that contacts the structure to the patient by means of an adhesive layer provided on the structure. The method further includes the step of causing thermal energy to be transferred into and out of the structure by adding heat to or removing heat from the thermal energy transfer portion not in contact with the patient via a heat source or cooling mechanism, respectively.

The instant invention furthermore relates to a method of therapeutically cooling the body temperature of the patient that includes the step of placing a structure substantially conformable to the shape of the body of the patient into contact with the patient, the structure being hermetically sealed and having a heat transfer portion in contact with the patient, a heat dissipation portion and a fluidized medium changeable between a liquid state and a gaseous state for moving heat from the patient absorbed by the heat transfer portion to the heat dissipation portion, and the step of working a cooling mechanism cooperatively with the heat dissipation portion to removing the heat at the heat dissipation portion.

### BRIEF DESCRIPTION OF THE FIGURES

The present invention will become apparent and the invention itself will be best understood with reference to the following description of the present invention taken in conjunction with the accompanying drawings, wherein:

FIG. 1 is an illustration of a patient covered by exemplar device, sheet or pad of the instant invention;

FIG. 2 is a side view of the patient, with the sheet or pad of the instant invention drawn in exaggerated dimensions for illustration purpose, placed on top of the patient;

FIG. 3 is a cross-section view of the exemplar device of the instant invention, drawn with exaggerated dimensions for the purpose of better illustrating the invention;

FIG. 4 is a top view of the exemplar flat flexible patient temperature regulating device of the instant invention having added thereto a cooling and/or heating circuit to enhance the dissipation and/or adding of heat from/to the temperature regulating device;

FIG. 5 is a plan view of another embodiment of the instant invention in which an air cover is placed over the exemplar flat flexible patient temperature regulating device of the instant invention to enhance the transfer of heat between the device and temperature controlled air;

FIG. 5a is an enlarged cross-sectional view of a portion of the embodiment of FIG. 5;

FIG. 6 is a plan view of an embodiment of the exemplar flat flexible device where the device is a configured as a rib shaped pad or jacket adaptable to be folded to embrace the torso of the patient;

FIG. 7 is a perspective view of the FIG. 6 jacket shown having its rib strips folded relative to its longitudinal support member into the shape of a rib cage;

FIG. 8 shows the FIG. 6 jacket with its rib strips contiguously and intimately embracing the torso of the patient; and

FIG. 9 shows an exemplar belt adapted to wrap around a limb of a patient that is made of a flexible heat pipe layer.

### DETAILED DESCRIPTION OF THE INVENTION

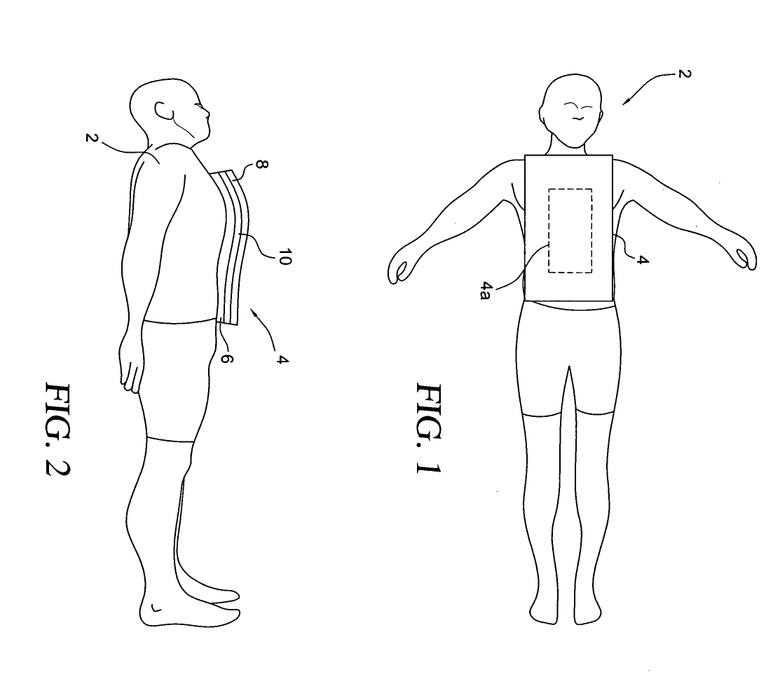

With reference to FIG. 1, a patient 2 who may be a stroke victim or has a medical condition such as for example hyperthermia that requires induced hypothermia is shown to have placed on him a therapeutic cooling device of the instant invention. Note however that the device may also be placed beneath the patient. The therapeutic device of the instant invention is a structure that may be a flat flexible heat pipe 4 that is conformable to the body of the patient. An example of such flat flexible heat pipe is described in U.S. Pat. No. 6,446,706, the disclosure of which is incorporated by reference herein. The heat pipe disclosed in the '706 patent is used to cool electronic equipment, as were most other heat pipes previously known. See for example U.S. Pat. No. 4,019,098 and U.S. publication 2006/0185827 which are directed to non-flat heat pipes. Another flat flexible heat pipe that may be used for the structure of the instant invention device is that made by the Furukawa Electric Company of Japan with the trade name TERA FLEX®.

With reference to FIG. 2, an exemplar flat flexible heat pipe 4, drawn with exaggerated dimensions for illustration purposes, is shown to have been placed over patient 2. The flat flexible heat pipe 4 has a first heat transfer portion 6 that is in contact with the body of the patient, and a second heat transfer portion 8 that is not in contact with the patient. Heat transfer portion 6 may be considered the heat absorption or vaporizer portion while heat transfer portion 8 may be considered the heat dissipation or condenser portion of the heat pipe pad, or sheet, of the instant invention. Although each of the heat transfer portions 6 and 8 is shown to extend along the length of the heat pipe pad, in practice the heat dissipation portion 8 may be located at a particular section of the pad or remotely located at a section of the pad not covering the patient. Thus, the heat dissipation portion may have any shape or be located anywhere on the pad so long as it does not come into contact with the patient and it has a dimension sufficient to carry away the heat absorbed from patient 2 by the heat transfer portion 6.

There is also provided in heat pipe sheet or pad 4 a layer portion 10 sandwiched between the two heat transfer portions 6 and 8. A fluidized medium such as for example water, ethanol, methanol or other similar fluid is stored in layer 10, after air has been evacuated from that portion of the structure. The fluidized medium is stored under vacuum within layer 10 and the heat pipe structure is hermetically sealed.

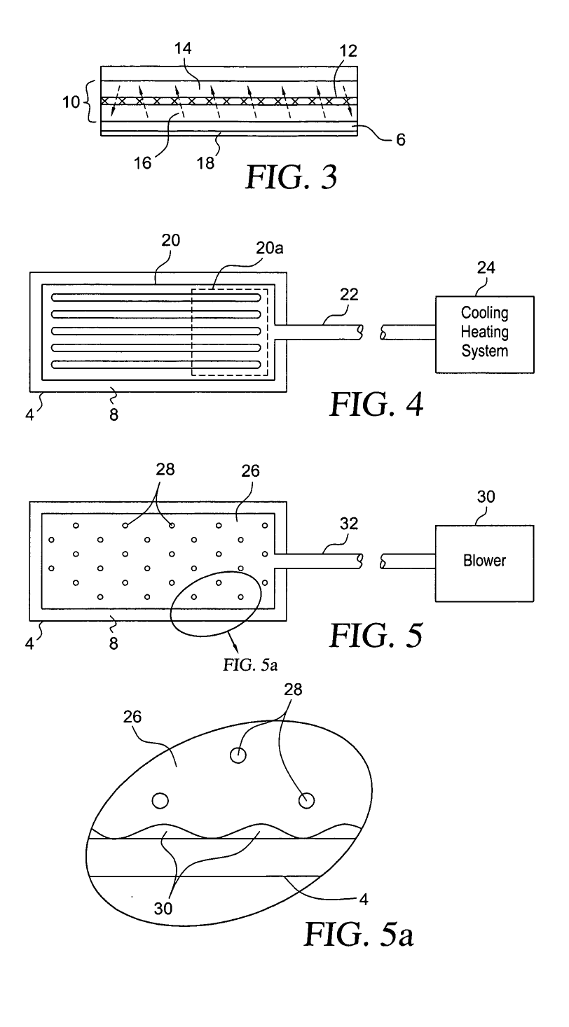

FIG. 3 provides a more detailed cross-sectional view of the exemplar flat flexible heat pipe patient pad or sheet of the instant invention which may be reflective of a heat pipe disclosed in the above noted '706 patent or the TERA FLEX® heat pipe. As illustrated, sandwiched between patient contact heat transfer portion 6 and heat transfer portion 8 that dissipates the heat from the pad is portion 10 that includes a wick layer 12. As exaggeratedly shown, wick layer 12 is separated from heat transfer portion layers 6, 8 spatially by spacer layers 14 and 16. With wick layer 12 and spacer layers 14 and 16, a space is provided in the heat pipe structure for the fluidized medium, i.e., the working fluid, to circulate or move the heat absorbed at heat transfer portion 6 to the heat dissipation portion 8.

Although the flat flexible heat pipe disclosed in the '706 patent and the TERA FLEX® heat pipe each are disclosed to have a wick layer, a flat flexible heat pipe that has a spatial portion or capillary layer that separates the two heat transfer portions and has a dimension sufficient to provide capillary pressure so that vaporized gas can condense at the heat transfer portion that acts as the condenser and return as a liquid to the heat transfer portion that acts as the evaporator portion may also be used.

An adhesive layer 18 is provided at the outer surface of heat transfer portion 6 to enable the pad to be fixedly attached or adhered to the patient when it is pressed onto the body of the patient, so that the pad would remain in contiguous and intimate contact with the patient. Having the heat transfer portion of the pad in contiguous and intimate contact with the patient ensures that there are no air pockets between the patient and the pad, thereby facilitating the transfer of the heat between the patient and the pad.

It should be appreciated that insofar as portion 6 and 8 each are a heat transfer portion, instead of removing heat from the patient, in those instances where the core body temperature of the patient needs to be raised, thermal energy may be applied to heat transfer portion 8, so that the external heat may be carried and transferred to the patient by heat transfer portion 6.

The reason that the heat pipe pad of the instant invention is able to effect heat or thermal energy transfer between the patient and the environment is because a heat pipe is a two phase heat transfer device with an extremely high effective thermal conductivity, and a lower total thermal resistance than solid conductors, that enables it to transfer heat more efficiently and evenly, thereby effecting an excellent isothermal condition. In addition, a heat pipe is a passive heat transfer system, in that as the structure illustrated in FIG. 4 is hermetically sealed, the only reaction is the internal physical phase changes between a liquid state and a gaseous state by the fluidized medium stored in layer or portion 10. Thus, there are no moving parts to wear out in the patient heat pipe pad of the instant invention. Moreover, the effective thermal conductivity of the exemplar flat flexible heat pipe utilized for the instant invention system may be up to 100 times better than copper, 500,000 times better than a polymer, or 100,000 times better relative to water.

The fluidized medium is back filled into the portion 10 of the pad 4 after air has been evacuated from portion 10. As was noted before, the fluidized medium may be water, ethanol, methanol, or some other fluid that is adapted to change from a liquid to a gas, or vaporized liquid, when exposed to heat. The heat is carried as latent heat in the vaporized liquid or heated gas. The heated gas condenses back into a liquid when it is chilled and the latent heat in the gas is removed.

In operation, in the case of induced hypothermia in a patient, the heat absorbed from the patient by the pad heats up the liquid in the pad, which is under vacuum, so that the liquid vaporizes. As the vaporized liquid, or heated gas vapor, transfers or moves to the heat dissipation or condenser portion of the structure, the latent heat in the gas is removed. As a result, the heated gas condenses back into a liquid. The condensed liquid then returns to the heat transfer portion by means of the capillary action of wick layer 2, and/or also by gravity, so that the condensed liquid once again gets evaporated by the heat at the heat transfer portion in contact with the patient so that the process of removing heat from the patient, i.e., inducing hypothermia to the patient, continues.

For the heat pipe patient pad 4 shown in FIG. 3, heat transfer portion layers 6 and 8 may be made of copper or some similar metal foil. Other polymer such as polyolefin layers may laminate the metal foils to add integrity to the pad.

To facilitate the removal of heat from the patient pad 4, an external temperature regulating system may be provided. This is shown in FIG. 4 where the patient pad 4 has on top thereof an active heat transfer layer 20, which may be an envelope like layer 20 that has a plurality of fluid passages and a fluid connect tube 22 that connects it to a cooling/heating system 24. System 24 may be a chiller, or a refrigerant cooling mechanism 24 when the device is used for induced hypothermia. Connect tube 22 has an inlet and an outlet, not shown for the sake of simplicity, that allow the chilled fluid, for example cooled water, to be sent from the chiller system 24 to layer 20 to pick up heat from the heat dissipation portion or layer of the pad and carry the dissipated heat to the chiller. As such chiller circuit is well known in the refrigeration or cooler art, nothing more needs to be said except that the chiller layer 20 may be permanently or removably attached or adhered to the top surface of the heat dissipation layer 8 of the pad 4. Moreover, instead of covering substantially the top of pad 4, layer 20 may actually be of a size that only covers a portion of the body of the patient, per shown by the dotted layer 20a. Chiller 24 therefore works cooperatively with the heat dissipation layer portion 8 of the heat pipe structure to enhance the removal of heat therefrom.

In the event that the core temperature of a patient needs to be increased, pad 4 may also be used. In that case, the temperature regulating system 24 becomes a heater that uses a heating fluid, for example heated water, that circulates from the heater to layer 20 to add heat to pad 4 to warm the patient. Therefore, a heating circuit may also work cooperatively with layer 20, where layer 20 becomes a heater layer.

In place of a chiller circuit, the removal of heat from pad 4 may be facilitated by the passing of cool air to the top surface of heat dissipation layer portion 8. This is shown in the embodiment of FIG. 5 where on top of layer 8 there is placed, or fixed thereto by adhesive means such as Velcro, an air layer 26. Layer 26, as shown in the enlarged view of a portion thereof in FIG. 5a, may be a cap that has a number of air holes or apertures 28 through which the pressurized air from a blower 30 fluidly connected by a connection tube 32 thereto may pass. Per shown in the cross-sectional view of FIG. 5a, at the lower portion of cap 26 there may also be openings 30 that allow additional air to pass through. The cooling air carries away from layer 8 the heat collected thereat. Blower 30 thus works cooperatively with the heat transfer layer portion 8 of the heat pipe to enhance the removal of heat therefrom. It should be appreciated that heated air may be output from blower 30 to layer 26 to add heat to pad 4 when normalization of the patient's body temperature is desired. Thus, the blower embodiment of FIG. 5 may be used to both remove and add heat to pad 4.

In operation, pad 4 is placed on top of the patient so that heat from the patient may be absorbed and removed. As shown in FIG. 1, pad 4 may be configured to have certain portions that may be removed, if induced hypothermia is desired. Conversely, as discussed above, if the core temperature of the patient needs to be raised, heat is added to the pad.

As shown in FIG. 1, pad 4 may be configured to have certain portions that may be removed such as for example portion 4a, so that the medical personnel such as a surgeon may operate on that body area of the patient while the patient continues to be covered by the heat pipe pad. Alternatively, different portions of the body of the patient may covered by different corresponding pads for selective cooling/heating of the different portions of the patient. In addition, the flat flexible heat pipe structure of the instant invention may be configured into shapes that allows it to be used to wrap around or wearable by the torso and/or the limbs such as for example the arms, legs of the patient for selective cooling/heating of the different parts of the body of a patient. The wrap around or wearable flexible heat pipes may be secured about the limb of a patient by securing means such as Velcro. Moreover, the heat pipe structure of the instant invention may also be formed into the shape of the head of the patient for directly transferring heat between the head of the patient and the heat pipe.

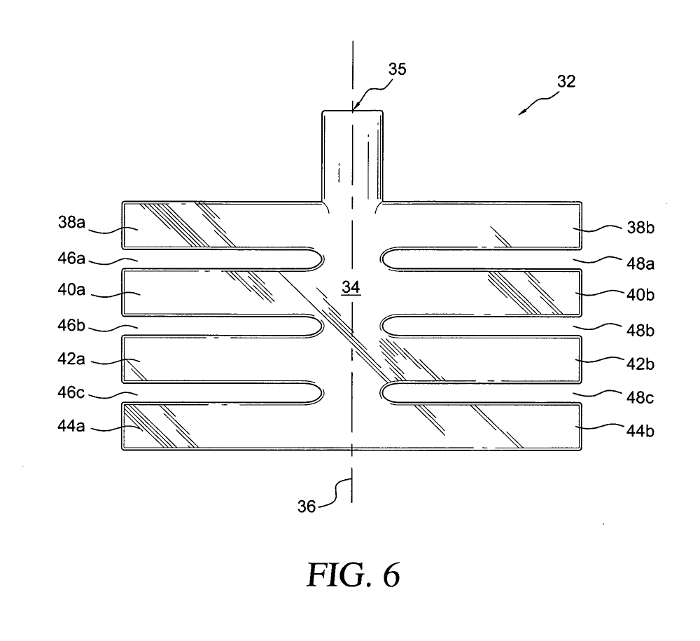

FIG. 6 illustrates an exemplar embodiment of a flexible pad 32 that is conformal or conformable to the contour of the torso of a patient. Pad 32 may be structurally constructed the same as the above discussed flat flexible heat pipes. In other words, it has the same heat transfer portions, wick layer and fluidized medium of the earlier discussed heat pipes, and operates to transfer heat between the patient and the pad in substantially the same manner as previously described.

For the instant invention, the pad may be referred to as a jacket or vest. In particular, in the plan view of FIG. 6, jacket 32 has a central support member 34 that extends longitudinally along a central axis 36. Extending perpendicularly or at right angle from the respective sides of member 34 are a plurality of ribs or strips 38a, 38b, 40a, 40b, 42a, 42b, and 44a, 44b. As shown, respective adjacent pairs of strips are separated spatially by corresponding slots. For example, strips 38a and 40a are separated by slot 46a, strips 40a and 42a by slot 46b, and strips 38b and 40b by slot 48a, etc. Other slots shown for the exemplar rib shaped jacket are 46c and 48b, 48c. The slots 46a-46c and 48a-48c separating the adjacent rib strips allow the rib strips to be foldable into the shape of a rib cage so that jacket 32 is conformable to the torso of a patient. Note that the dimension of the slots are not drawn to scale, as the slots may in practice be simply a line cut that separates adjacent rib strips.

There is also provided at jacket 32 a connector 35 that acts as an interface to connect the jacket to a temperature regulating system such as for example the cooling heating system 24 shown in FIG. 4 via conduit 22. Although not shown, on the heat transfer portion of the jacket that is not in contact with the patient there is a layer or portion wherethrough the cooling or heating fluid such as water from system 24 may be circulated, so that heat may be removed or added to that heat transfer portion for cooling the patient or raising the core temperature of the body of the patient, respectively.

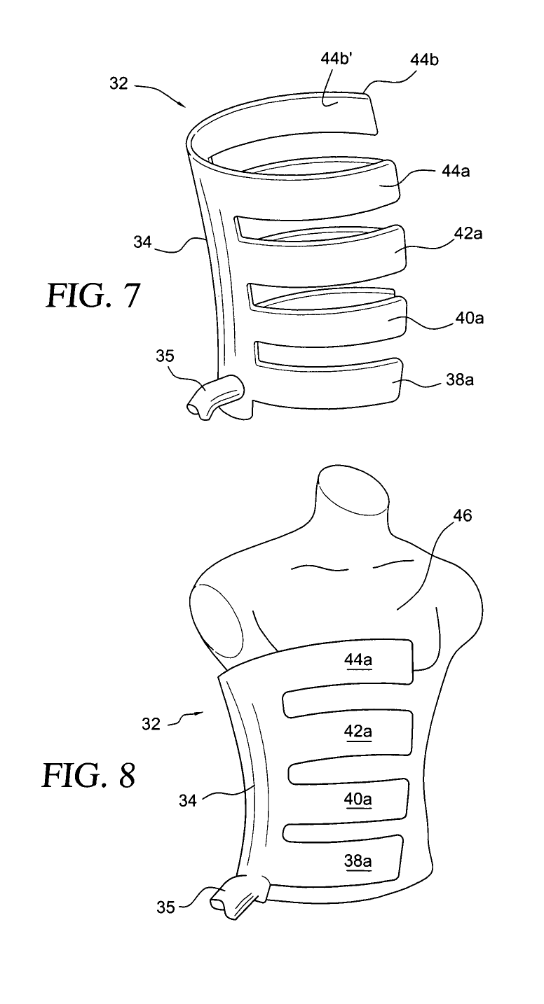

To ensure contiguous and intimate contact between the patient and the surface of jacket 32 that comes into contact with the patient, the respective inner surfaces of the rib strips 38-44, for example surface 44b′ shown in FIG. 7, and the support member 34 are coated with an adhesive layer as discussed with the earlier embodiments.

With reference to FIG. 7, jacket 32 is shown with its rib strips 38-44 folded to form a rib cage structure to embrace the torso of a patient.

FIG. 8 shows jacket 32 embracing torso 46 of the patient from the right hand side of the patient. It should be appreciated that jacket 32 is equally adaptable to embrace the torso of the patient from his left side. Although shown not fully circumferentially embracing the torso of the patient, in practice the respective lengths of the rib strips 38-44 may be such that the entire torso of the patient is encircled. In other words, the lengths of the strips vary depending on the size of the jacket, which in turn depends on the size of the patient. For example, a normal size patient would use regular jacket whereas an obese patient would need to use a large jacket with strips having lengths sufficient to embrace the torso of the patient. To fully secure jacket 32 to the patient to effect enhanced intimate contact between the skin of the patient and the rib strips of the jacket, secure means such as Velcro may be added to the end portions of the different rib strips so that matching rib strips, for example 44a and 44b, that extend from opposed sides of support member 34 may be secured to each other. It should be appreciated that when the secure means are used to secure the rib strips, there is no need for an adhesive layer for the strips.

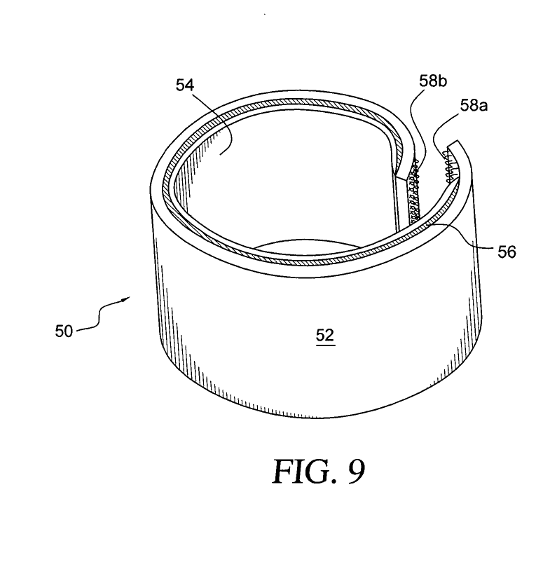

Although the rib cage shaped heat pipe in the FIGS. 7 and 8 embodiment is referred to as a jacket or vest, there may be instances where a flat flexible heat pipe may be manufactured as a temperature regulating portion of a belt, jacket or vest made of conventional material for wrapping around given portions of the body of a patient. Such combination heat pipe and belt may therefore simply be referred to as a wrap. One such exemplar wrap is belt 50 illustrated in the perspective cross sectional view of FIG. 9. As shown, belt 50 includes an outer layer 52 that is made of a conventional material such as leather, fabric or plastic. A flat flexible heat pipe forms the inner layer 54 of belt 50. To attach the heat pipe inner layer 54 to the outer layer 52, a layer of compliant or compressible foam 56 with adhesive on both its sides is placed between layers 52 and 54. Foam layer 56 serves two purposes: one, to attach the inner heat pipe layer 54 to the outer surface layer 52, and two, to make the heat pipe layer that contacts the skin of the patient to be more compliant so as to reduce the chances that air pockets may be formed between the skin of the patient and the heat pipe. There is also formed at the respective ends of the belt 50 secure means such as for example a Velcro strap 58a and loops 58b to securely wrap the belt about a limb such as for example an upper leg of a patient. Although a foam layer with adhesive layers on both of its sides is disclosed as the means for attaching the heat pipe to the wrap in the FIG. 9 embodiment, in practice the heat pipe may be attached directly to the outer layer 52, such as for example by directly glued or fastened thereto.

1. A device placeable in contact with a patient for therapeutically cooling the patient, comprising:
a structure substantially conformable to the shape of at least one part of the body of the patient, said structure being hermetically sealed and having at least one heat transfer portion in contact with the patient, at least one heat dissipation portion, and a fluidized medium changeable between a liquid state and a gaseous state provided in said structure for moving the heat transferred from the patient to said heat transfer portion to said heat dissipation section for removal; and
an adhesive layer provided on the patient contact surface of said structure to ensure that said structure is in contiguous contact with the patient.

2. The device of claim 1, wherein said structure comprises a substantially flat flexible heat pipe having a dimension adapted to cover at least one part of the body of the patient.

3. The device of claim 1, wherein said structure comprises a flat flexible heat pipe configured to have a central longitudinal portion having extending substantially perpendicularly from both sides thereof a plurality of strips, each of said strips being foldable about the torso of the patient so that said heat pipe is conformable to the contour of the torso of the patient.

4. The device of claim 3, further comprising secure means for securing said heat pipe in contiguous contact with the patient.

5. The device of claim 1, further comprising:
cooling means to remove the heat from said heat dissipation portion, wherein said cooling means comprises a mechanism for circulating cooling air over said heat dissipation portion to remove heat therefrom.

6. The device of claim 1, further comprising a chiller circuit working cooperatively with said heat dissipation portion to circulate a cooling fluid to said heat dissipation portion to remove heat from said heat dissipation portion.

7. The device of claim 2, wherein said heat pipe is formed in the shape of a pad or a sheet adaptable to be positioned on top or underneath a patient, and also wrap around or wearable by the torso or a limb of the patient.

8. A therapeutic device placeable into contact with a patient for regulating the body temperature of the patient, comprising:
a structure substantially conformable to the shape of at least one part of the body of the patient, said structure being hermetically sealed and having a first thermal energy transfer portion in contact with the patient, a second thermal energy transfer portion, and a fluidized medium within said structure changeable between a liquid state and a gaseous state for carrying heat absorbed from one of the thermal energy transfer portions to the other of the thermal energy transfer portions; and
an adhesive layer provided on the patient contact surface of said structure to ensure that at least said first energy transfer portion of said structure remains in contiguous contact with the patient.

9. The device of claim 8, wherein one of the thermal energy transfer portions comprises a heat absorption portion and the other of the thermal energy transfer portions comprises a heat dissipation portion.

10. The device of claim 8, wherein said heat absorption portion of said structure is in contact with the patient so that heat from the patient is transferred to said heat absorbing portion.

11. The device of claim 8, wherein said structure comprises a substantially flat flexible heat pipe having an area of sufficient dimension in contact with the patient to enable an effective amount of heat to be transferred between the structure and the patient to regulate the body temperature of the patient.

12. The device of claim 8, wherein said heat dissipation portion is in contact with the patient so that heat may be transferred from a heat source to the patient.

13. The device of claim 8, further comprising:
a cooling mechanism working cooperatively with one of the thermal energy transfer portions to cool the one thermal energy transfer portion to remove heat from said structure.

14. The device of claim 8, wherein said structure comprises a flat flexible heat pipe; and wherein said first thermal energy transfer portion comprises a heat absorbing portion and said second thermal energy transfer portion comprises a heat dissipation portion.

15. The device of claim 8, wherein said structure comprises a flat flexible heat pipe configured to have a central longitudinal portion having extending substantially perpendicularly from both sides thereof a plurality of strips, each of said strips being foldable about the torso of the patient so that said heat pipe is conformable to the contour of the torso of the patient.

16. A system for therapeutically regulating the body temperature of a patient, comprising:
a structure substantially conformable to the shape of at least one part of the body of the patient, said structure being hermetically sealed and having at least one heat transfer portion in contact with the patient, at least an other heat transfer portion and a fluidized medium changeable between a liquid state and a gaseous state for moving the heat absorbed at one of said one and other heat transfer portions to other of said one and other heat transfer portions for removal; and
an adhesive layer provided on the patient contact surface of said structure to ensure that at least said one heat transfer portion remains in contiguous contact with the patient.

17. System of claim 16, further comprising:
a cooling mechanism working cooperatively with said other heat transfer portion for removing heat collected thereat.

18. System of claim 16, further comprising:
a heat source working cooperatively with said other heat transfer portion for adding heat thereto.

19. The system of claim 16, wherein said structure comprises a flat flexible heat pipe adaptable to be shaped to conform to the part of the body of the patient; and wherein said one heat transfer portion comprises an area of sufficient dimension in contact with the patient to enable an effective amount of heat to be transferred between said structure and the patient to thereby therapeutically control the body temperature of the patient.

20. The device of claim 16, wherein said structure comprises a flat flexible heat pipe configured to have a central longitudinal portion having extending substantially perpendicularly from both sides thereof a plurality of strips, each of said strips being foldable about the torso of the patient so that said heat pipe is conformable to the contour of the torso of the patient.

21. A method of therapeutically regulating the body temperature of a patient, comprising the steps of:
placing a structure substantially conformable to the shape of at least one part of the body of the patient into contact with the patient, said structure being hermetically sealed and having a first thermal energy transfer portion that comes into contact with the patient, a second thermal energy transfer portion, and a fluidized medium changeable between a liquid state and a gaseous state within said structure for carrying heat absorbed from one of the thermal energy transfer portions to the other of the thermal energy transfer portions;
adhering at least said first thermal energy transfer portion that contacts the patient to the one part of the patient by means of an adhesive layer provided on said structure; and
causing thermal energy to be transferred into or out of said structure by adding heat to or removing heat from said second thermal energy transfer portion of said structure via a heat source or a cooling mechanism, respectively.

22. The method of claim 21, further comprising the step of:
forming said structure from a substantially flat flexible heat pipe adaptable to conform to the shape of or wearable around the one part of the body of the patient, the one part being the torso and/or any of the limbs of the patient.

23. A method of therapeutically cooling the body temperature of a patient, comprising the steps of:
placing a structure substantially conformable to the shape of at least one part of the body of the patient into contact with the patient, said structure being hermetically sealed and having a heat transfer portion in contact with the patient, a heat dissipation portion and a fluidized medium changeable between a liquid state and a gaseous state for moving the heat from the patient transferred to said heat transfer portion to said heat dissipation portion; and
working a cooling mechanism cooperatively with said heat dissipation portion for removing heat thereat.

24. Method of claim 23, further comprising the step of:
adhering at least said heat transfer portion of the structure to the patient by means of an adhesive layer provided on said structure.

25. Method of claim 23, said method further comprises the step of:
forming said structure from a substantially flat flexible heat pipe adaptable to conform to the shape of the one part of the body of the patient.

26. A jacket conformable to the shape of at least the torso of a patient for regulating the body temperature of the patient, comprising:
a rib shaped hermetically sealed structure having a first thermal energy transfer portion in contact with the patient, a second thermal energy transfer portion, and a fluidized medium within said structure changeable between a liquid state and a gaseous state for carrying heat absorbed from one of the thermal energy transfer portions to the other of the thermal energy transfer portions; and
secure means to secure said jacket to the patient such that contiguous contact is effected between substantially all ribs of said structure and the body of the patient.

27. Jacket of claim 26, wherein said structure comprises a flat flexible heat pipe configured to have a central longitudinal portion having extending substantially perpendicularly from both sides thereof a plurality of strips, each of said strips being foldable about the torso of the patient so that said heat pipe is conformable to the contour of the torso of the patient.

28. Jacket of claim 26, further comprising:
an interface at said structure connectable to a temperature regulating system, wherein said temperature regulating system operates cooperatively with said structure for circulating a cooling fluid to said second thermal energy transfer portion to remove heat therefrom or a heating fluid to said second thermal energy transfer portion to add heat thereto.

29. Jacket of claim 28, wherein adjacent strips of said structure are separated spatially so that the strips each are foldable about the torso of the patient.

30. A wrap adapted the wrap around a limb of a patient for regulating the temperature of at least the limb the patient, comprising:
a hermetically sealed structure having a first thermal energy transfer portion in contact with the patient, a second thermal energy transfer portion, and a fluidized medium within said structure changeable between a liquid state and a gaseous state for carrying heat absorbed from one of the thermal energy transfer portions to the other of the thermal energy transfer portions;
an elongate outer structure to which said sealed structure is attached; and
secure means at at least one end of said elongated structure to enable said wrap to be securely wrap about the limb of the patient such that contiguous contact is effected between said sealed structure and the limb of the patient.

31. Wrap of claim 30, further comprising:
a compliant foam layer having adhesive on both of its side interposed between said sealed and elongate structures for attaching said structures together.

32. Wrap of claim 31, wherein said structures attached together by said foam layer are configured into a belt.
